(19) United States
(12) Patent Application Publication
(10) Pub. No.: US 2008/0051732 A1
(43) Pub. Date: Feb. 28, 2008

(54) DROP SENSING DEVICE FOR MONITORING INTRAVENOUS FLUID FLOW

(76) Inventor: Thaiping Chen, Kaohsiung City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/426,106

(22) Filed: Jun. 23, 2006

(51) Int. Cl. A61M 5/14; A61M 5/00

(52) U.S. Cl. 604/253; 604/251

(57) ABSTRACT

A drop sensing device for monitoring intravenous (IV) fluid flow is provided. The device comprises a U-shaped clip mountable on a drip chamber of an IV infusion set. The clip has a light source provided at one side for emitting radiation and a hollow cylinder provided in a through hole on the side opposite the light source. The cylinder includes an aperture of a diameter smaller than an average diameter of fluid drops passing the drip chamber, and an optical sensor mounted on a rear end of the cylinder opposite the aperture, so as to substantially enhance sensitivity of light ray receiving.

### BACKGROUND OF THE INVENTION

1. Field of Invention

The present invention relates to drop sensing devices for monitoring intravenous (IV) fluid flow and more particularly to an optical sensor for such drop sensing devices for reliably sensing drops passing through a drip chamber of an IV infusion set by greatly increasing light ray receiving to enhance drop sensing sensitivity.

2. Related Art

A conventional device for sensing drops of IV fluid flowing through a drip chamber 40 of an IV infusion set is shown in FIG. 1. The device is implemented as a U-shaped clip 10 fastened on the drip chamber 40. The U-shaped clip 10 comprises a light source 20 provided in a through hole on one side and an optical sensor 30 provided in a through hole on the opposite side. Light ray produced by the light source 20 should be brighter than light in the environment. The light source 20 is adapted to send light ray along a path (i.e., a straight line from the light source 20 to the optical sensor 30) toward the optical sensor 30. When a fluid drop falls and crosses the path, the absence of the light ray being received at the optical sensor 30 provides an indication that the dripping is normal. To the contrary, it is determined that the dripping has stopped if the light ray is continuously received by the optical sensor 30 within a predetermined period of time. And in turn, an alarm system is immediately activated by the IV infusion set so as to audibly and/or visually inform a medical worker to take appropriate actions.

However, the optical sensor 30 of the prior-art sensing device may be excessively sensitive and still can receive the light ray even if a drop regularly crosses the path, and thus indicates no drop passing. It is thus erroneously determined that the dripping has stopped after the predetermined period of time has elapsed. This is not desirable. Hence, a need has arisen for an improved device for correctly sensing IV fluid.

### SUMMARY OF THE INVENTION

It is therefore an object of the present invention to provide a beam-path type U-shaped clipping device for correctly sensing fluid drops passing through a drip chamber of an IV infusion set by greatly decreasing light ray receiving sensitivity in case a drop passes through the drip chamber and greatly increasing light ray receiving sensitivity in case no drop passes through same.

To achieve the above and other objects, the present invention provides a drop sensing device for monitoring intravenous (IV) fluid flow, comprising a U-shaped clip mountable on a drip chamber of an IV infusion set and having a light source provided at one side for emitting radiation; wherein a hollow cylinder is provided in a through hole on the side opposite the light source and includes an aperture through a front end aligned with the light source, the aperture having a diameter smaller than an average diameter of fluid drops passing the drip chamber, and an optical sensor mounted on a rear end of the cylinder opposite the aperture.

In one aspect of the present invention the cylinder further comprises an internal guide formed around the inner mouth of the aperture for substantially increasing strength of light ray receiving from the light source.

In another aspect of the present invention an inner surface of the cylinder is plated with a reflective layer for increasing reflection of light ray entering the aperture so as to enhance sensitivity of light ray receiving.

The above and other objects, features and advantages of the present invention will become apparent from the following detailed description taken with the accompanying drawings.

### BRIEF DESCRIPTION OF THE DRAWINGS

FIG. 1 is a longitudinal sectional view of a conventional beam-path type U-shaped device mounted on a drip chamber for sensing IV dripping;

FIG. 2 is an exploded view of a preferred embodiment of a device for sensing fluid flow according to the invention;

FIG. 3 is a longitudinal sectional view of the device of FIG. 2 mounted on a drip chamber for sensing IV dripping;

FIG. 4 is a transverse sectional view of FIG. 3; and

FIG. 5 is a perspective view of the assembled device of FIG. 2 to be mounted on a drip chamber of an IV infusion set.

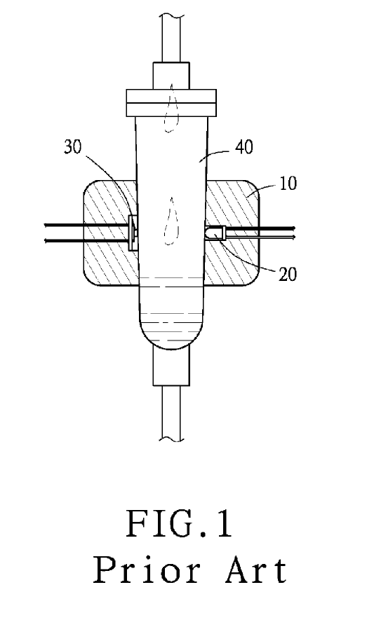

### DETAILED DESCRIPTION OF THE INVENTION

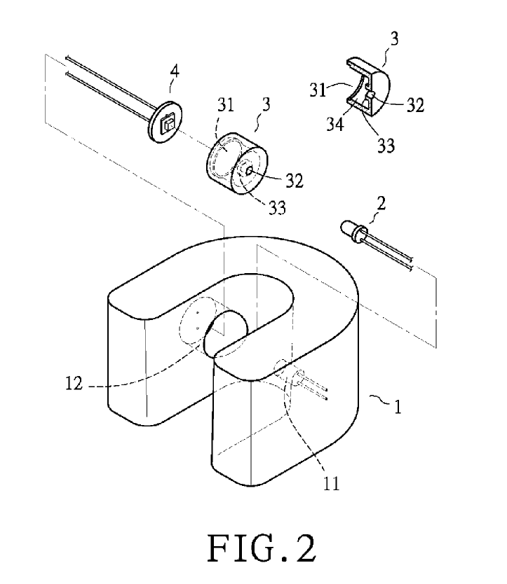

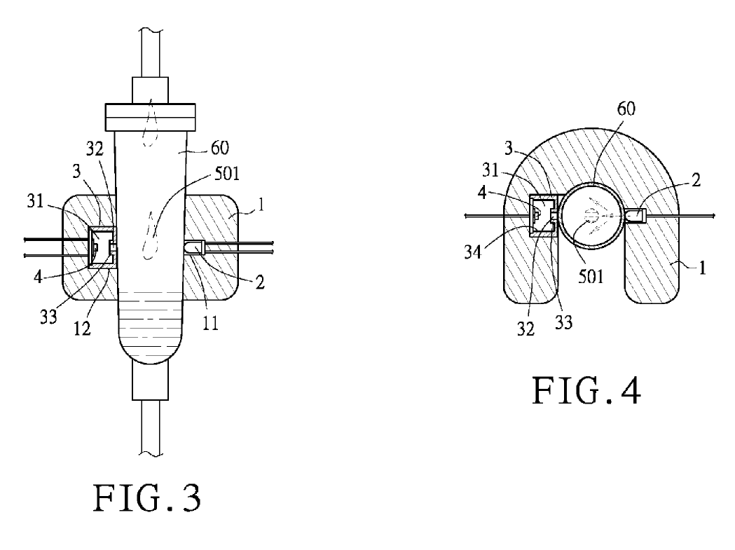

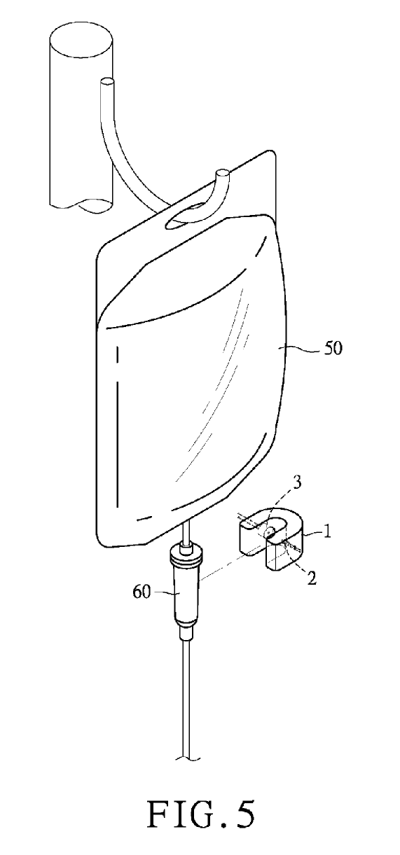

Referring to FIGS. 2 to 5, a drop sensing device for monitoring intravenous (IV) fluid flow for an IV infusion set is shown. The device includes a U-shaped clip 1 mounted on a drip chamber 60 of the IV infusion set. The IV infusion set further comprises an IV bag containing IV fluid 50. The IV fluid bag is in fluid communication with the drip chamber 60 as well known in the art. The U-shaped clip 1 includes a light source 2 provided in a through hole 11 on one side, and a hollow cylinder 3 provided in a through hole 12 on the opposite side. The light source 2 is preferably implemented as a light resistor, LED (light-emitting diode) or photonic crystal. The cylinder 3 comprises an internal space 31 and is plated with a reflective layer 34 on its inner surface. An optical sensor 4 can be provided inside the space 31. An aperture 32 is provided on one end of the cylinder 3 opposite the optical sensor 4 and has a diameter much smaller than the size of a fluid drop. A guide ring 33 is formed in the space 31 around an inner month of the aperture 32.

The light source 2 is adapted to send light ray along a path (i.e., a straight line from the light source 2 to the optical sensor 4 through the aperture 32) toward the optical sensor 4. Advantageously, the light ray passing through the aperture 32 is further guided to the optical sensor 4 by the guide ring 33 if no drop passes through the path. Furthermore, the light ray is focused on one face of the optical sensor 4 due to the reflection of the reflective layer 34. Thus, a signal representing the light ray received by the optical sensor 4 is strong enough to determine that there is no drop passing through the drip chamber 60.

To the contrary, when a drop 501 in the drip chamber 60 crosses the path, the absence of the light ray being received at the optical sensor 4 provides an indication that IV dripping is normal. Note that the light ray strength is much weakened by the drop 501 (i.e., substantially completely dispersed in the drip chamber 60) when they cross each other. Thus, the light ray received by the optical sensor 4 is very weak after passing through the aperture 32. Hence, the absence of the light ray determined by the optical sensor 4 in case of drop passing is correct and reliable.

It is determined that the IV dripping has stopped if the light ray is continuously received by the optical sensor 4 within a predetermined period of time. And in turn, an alarm system is immediately activated by the IV infusion set so as to audibly and/or visually inform a medical worker to take appropriate actions.

In brief, as envisaged by the invention the determination of whether IV dripping is normal or not is very reliable by greatly decreasing light ray receiving sensitivity in case an IV drop passes through a drip chamber and greatly increasing light ray receiving sensitivity in case no IV drop passes through same.

While the invention herein disclosed has been described by means of specific embodiments, numerous modifications and variations could be made thereto by those skilled in the art without departing from the scope and spirit of the invention set forth in the claims.

What is claimed is:

1. A drop sensing device for monitoring intravenous (IV) fluid flow, comprising a U-shaped clip mountable on a drip chamber of an IV infusion set and having a light source provided at one side for emitting radiation; wherein a hollow cylinder is provided in a through hole on the side opposite the light source and includes an aperture through a front end aligned with the light source, the aperture having a diameter smaller than an average diameter of fluid drops passing the drip chamber, and an optical sensor mounted on a rear end of the cylinder opposite the aperture.

2. The drop sensing device of claim 1, wherein the cylinder further comprises an internal guide formed around the inner mouth of the aperture for substantially increasing strength of light ray receiving from the light source.

3. The drop sensing device of claim 1, wherein an inner surface of the cylinder is plated with a reflective layer for increasing reflection of light ray entering the aperture so as to enhance sensitivity of light ray receiving.
